(19) United States
(12) Patent Application Publication
(10) Pub. No.: US 2019/0083516 A1
(43) Pub. Date: Mar. 21, 2019

(54) NUTRITIONAL SUPPLEMENT FOR INCREASING CELLULAR LEVELS OF N-ACETYLNEURAMINATE

(71) Applicant: VENTRINE S. DE R.L. DE C.V., Mexico D.F. (MX)

(72) Inventors: Yadira VALLES-AYOUB, Woodland Hills, CA (US); Daniel DARVISH, Chatsworth, CA (US); Carlos Asdrubal VALLES ORTIZ, Mexico, D.F. (MX); Sergio Jaime ARIAS ZEPEDA, Jalisco (MX)

(73) Assignee: VENTRINE S. DE R.L. DE C.V., Mexico D.F. (MX)

(21) Appl. No.: 15/527,470

(22) PCT Filed: Nov. 18, 2015
(86) PCT No.: PCT/IB2015/058932

§ 371 Date: May 17, 2017

Related U.S. Application Data
(60) Provisional application No. 62/081,843, filed on Nov. 19, 2014.

(51) Int. Cl. A61K 31/7012; A61K 31/702; A61K 31/7004; A61K 33/06; A61K 9/16; A23L 33/10

(52) U.S. Cl. CPC A61K 31/7012; A61K 31/702; A61K 31/7004; A23V 2002/00; A61K 9/1652; A23L 33/10; A61K 33/06

(57) ABSTRACT

The instant invention relates to a composition useful as a nutritional supplement comprising acetylated neuraminate (NeuAc), and/or a compound selected from N-acetyl mannosamine (ManNac), at least one or more compounds of the NeuAc biosynthetic pathway, substrates, products, or derivative thereof, or a mixture thereof; and a divalent metal ion. The composition of the invention permits to increase serum and or cellular NeuAc content in a subject.

### TECHNICAL FIELD

The present invention is directed to a composition comprising acetylated neuraminate (NeuAc) and a metal divalent ion. Optionally, the composition may further comprise a compound selected from N-acetyl mannosamine (ManNac), at least one compound of the acetylated neuraminate biosynthetic pathway, or a derivative thereof, or a mixture thereof, for increasing, complementing or supplying acetylated neuraminate in a subject in biological system. The invention is also related to compositions comprising at least one compound of the acetylated neuraminate biosynthetic pathway such as sialyllactose, or a derivative thereof, or a mixture thereof, and a metal divalent ion for increasing, complementing or supplying acetylated neuraminate in a subject in biological system.

### BACKGROUND

N-Acetylneuraminate (NeuAc) occurs naturally in the human body and is a sugar that contains a net negative charge and is typically found on terminating branches of N-glycans, O-glycans, and glycosphingolipids (gangliosides), including occasional capping side chains of GPI anchors. The NeuAc modification of cell surface molecules is crucial for many biological phenomena including protein structure and stability, regulation of cell adhesion, and signal transduction.

The cellular production of NeuAc on glycoconjugates requires the conversion of N-acetylglucosamine (conjugated to its carrier nucleotide sugar UDP). NeuAc subsequently enters the nucleus where it is conjugated with its nucleotide sugar carrier CMP to make CMP-NeuAc, which is used as a donor sugar for glycosylation reactions in the cell. CMP-NeuAc is a known regulator of UDP-N-Acetylglucosamine-2-Epimerase activity. Jay et al., Gene Reg. & Sys. Biol. 3:181-190 (2009).

NeuAc deficiency is related to some disorders in man such as Distal Myopathy with Red Rimmed Vacuoles. Subjects may suffer difficulties walking with foot drop, gripping and using their hands, and normal body functions like swallowing. Additionally, beta-amyloid deposits in senile plaques of subjects with Alzheimer's brain disease may become more soluble with addition of increased amounts of NeuAc.

On the other hand, neuropathy also known as periphery neuropathy or polyneuropathy is related to nerve damage in the peripheral nervous system, particularly automatic nerves, motor nerves and sensory nerves. Neuropathy is a complication existing in diseases such as Diabetes, Chronic kidney disease, Chronic liver disease, Long-term excessive alcohol intake, Vitamin B Deficiency and other nutritional deficiencies, etc. (Information available at MNT website: http://www.medicalnewstoday.com/articles/147963.php)

Acetyled neuraminate could also be found in nature as an important component of high nutritional crude supplements, including but not limited to, “colostrum” and “edible bird's nest”. These crude nutritional food products contain relatively high amounts of NeuAc. They have been used for thousands of years to improve the nutritional status of both human athletes and farm animals, including prized racing/sports animals (horses, dogs, etc.).

Most NeuAc found in food and supplements is not pure, having varying amounts of N-acetylglycolyl (NeuGc). Humans make antibodies to NeuGc and it has been suggested that chronic ingestion of NeuGc can lead to subclinical inflammation in internal organs and vessels. Foods high in NeuAc, and lacking NeuGc, such as “edible bird's nest” has not been associated or reported with similar findings. Additionally, since the NeuAc biochemical pathway and glycosylation pathway in humans uses both NeuAc and NeuGc, high use of NeuAc can wash out or limit the incorporation of NeuGc in glycans, and may lead to more effective “washout” of NeuGc from the body.

NeuAc has been associated in numerous biological processes. NeuAc is required for robust embryogenesis, brain development, robust immune system, robust autophagy and lysosomal function, and maybe beneficial for muscle health, skin turgor and sheen, and hair sheen and strength.

High intake of NeuAc by pets and farm animals may benefit the animals in similar manner as described above, and can be specifically important for keeping nutritionally healthy farm animals and racing/sports animals. High NeuAc content in milk, meat, and chicken may be produced by animals that chronically ingest large amounts of NeuAc. Inventors anticipate that NeuAc rich food sources can also nutritionally benefit the people who ingest these high NeuAc foods and dietary supplements described here-in.

In spite of the listed advantages, Oral NeuAc is not well absorbed from Gastro Intestinal (GI) track for several reasons. The mouth/GI bacterial flora may breakdown NeuAc. The stomach acid low pH can breakdown significant portion of oral NeuAc, and either active or passive absorption mechanism in the intestine is not optimal for NeuAc (sugar of 9 carbons and negative charge).

Moreover, kidney filters NeuAc in similar manner as creatinine. On the other hand, divalent metals such as magnesium or calcium are involved in numerous biological processes. Magnesium is widely used both as dietary supplement and in significantly larger quantities as a medical treatment with wide therapeutic index. In addition to its standard use as therapy for specific cardiac arrhythmias, and eclampsia of pregnancy, magnesium has been advocated as adjunctive treatment for disorders ranging from sudden infant death syndrome, various cardiovascular disorders, and depression amongst others.

Therefore, in view of the biological importance of NeuAc, there is a need in the art for compositions useful as nutritional supplements having improved NeuAc absorption and bioavailability features.

### BRIEF DESCRIPTION OF THE INVENTION

The present invention provides a composition comprising acetylated neuraminate (NeuAc) and a divalent metal ion. Optionally, the composition may further comprise a compound selected from N-acetyl mannosamine (ManNac), a compound of the NeuAc biosynthetic pathway, substrates, products, or derivatives thereof, or a mixture thereof.

In a preferred embodiment, the divalent metal ion is preferably selected from magnesium.

In a preferred embodiment, the composition comprises at least one or more compounds of the NeuAc biosynthetic pathway and a divalent metal ion.

In other preferred embodiment, said at least one or more compounds of the NeuAc biosynthetic pathway could be selected, for instance, from the group comprising sialyllactose and other sialylated glycans, with or without additional acetyl groups on either ManNAc, NeuAc, or glycans rich in ManNAc or NeuAc.

In a preferred embodiment of the invention, the composition comprises acetylated neuraminate (NeuAc) and at least one compound of the NeuAc biosynthetic pathway; and a divalent metal ion, wherein said at least one compound of the NeuAc biosynthetic pathway is preferably selected from sialyllactose.

In another preferred embodiment, the composition comprises siallylactose and a divalent metal ion.

In some embodiments, the compositions of the invention may further comprise one or more of the following: a flavoring agent, a sweeter agent, a diluent, a pH adjuster, stabilizers, etc.

Certain embodiments of the inventions include, but are not limited to compositions useful as a nutritional supplement for a subject not receiving adequate amount of NeuAc in food or consuming a NeuAc poor-diet.

In other embodiments, the compositions of the invention are useful for providing a benefit to a human or animal subject for maintaining optimal health and of general well being, including but not limited to, gastrointestinal, immune, cardiovascular, integumentary, musculoskeletal, endocrine, or nervous systems.

In preferred embodiments, the invention provides a nutritional supplement comprising the compositions of the invention for providing nutritional benefits to a subject.

In some embodiments, the nutritional supplement comprising the composition of the invention are useful for increasing, complementing or supplying Acetylated Neuraminate in a subject having an average or higher than average consumption of NeuAc rich foods, but that has poor bio-availability due to either absorption limitations or limitations imposed by the normally poor absorption of free form of NeuAc.

In another embodiment, the nutritional supplement of the invention may offer additional nutritional benefits for people suffering from a disease or disorder related to glycosylation deficiencies or associated with sub-optimal glycosylation or a complication related to neuropathy, a kidney disorder or disease, blood pressure disorder such as hypertension or hypotension, etc. Such group may be comprised of subjects suffering from both common and rare/orphan disorders including, but not limited to, neurological disorders, neurodegenerative disorders, cognitive impairment or behavior, nutritional deterioration, disorders associated with cachexia and sarcopenia, some infectious disorders, and immune dysregulation, male infertility or reduced fertility, and female infertility or reduced female fertility.

In preferred embodiment, the compositions and nutritional supplements of the invention may be used by various subject groups including the elderly, young children, teenagers, adults, males and females.

In preferred embodiments, the compositions could be prepared, for instance, in the following forms: hydrophobic, liposomal, microsomal, nanoparticulates, powders, granules and suspensions.

In some embodiments, the invention provides methods for preparing the compositions of the invention.

In some embodiments, the liposomal composition comprises liposomes selected from the group of MLV (Multilamellar vesicles), SUV (Small unilamellar vesicles), LUV (Large unilamellar vesicles), GUV (Giant Unilamellar Vesicles), MVV (Multivesicular vesicles).

In certain embodiments of the invention compositions have a slow, controlled, extended, and sustained dissolution or release of active ingredients in intestine to keep blood NeuAc levels high throughout the 24 hours.

In some embodiments, the compositions of the instant invention are coated to protect against both bacterial and stomach acid breakdown.

In preferred embodiments, the composition provides to a subject from about 0.001 mg to 15 g/day (as single or multiple dosing per day) of NeuAc. Said levels refer to the serum levels of NeuAc after the composition or nutritional supplement intake by a subject.

In some embodiments and depending on factors such as subject's age, subject's state of health, etc. the composition provides from about 0.001 mg to 12 g/day, preferably, 0.001 mg to 6 g/day, more preferably, 0.0.001 mg to 1 g/day of NeuAc.

In preferred embodiments, the invention provides a nutritional supplement comprising at least one of the compositions previously disclosed.

In some embodiments, the nutritional supplement improves or supports the neurological, brain, nerve, cognitive, kidney, muscle, cellular, lysosomal, glycosylation function.

In additional embodiments, the nutritional supplement of the invention provides benefits to subjects suffering a complication due to a neuropathy, hypertension or hypotension or a kidney disorder such as those related to Diabetes.

In preferred embodiments, the invention provides methods to prevent or treat a nutritional deficiency related to low levels of NeuAc in a subject comprising administering an effective amount of a nutritional supplement comprising the composition of the invention to a subject in need thereof.

In some embodiments, the nutritional supplement comprising the composition of the invention permits to increase serum and or cellular NeuAc content in man and various animals (including but not limited to farm animal, pets, and zoo kept).

### BRIEF DESCRIPTION OF DRAWINGS

These and other features of the invention will be more readily understood from the following detailed description of the various aspects of the invention taken in conjunction with the accompanying drawing that depicts various embodiments of the invention:

FIG. 1 illustrates the NeuAc concentration in blood for a control group.

FIG. 2 illustrates the NeuAc concentration in blood for a group, wherein the composition of the invention was administered.

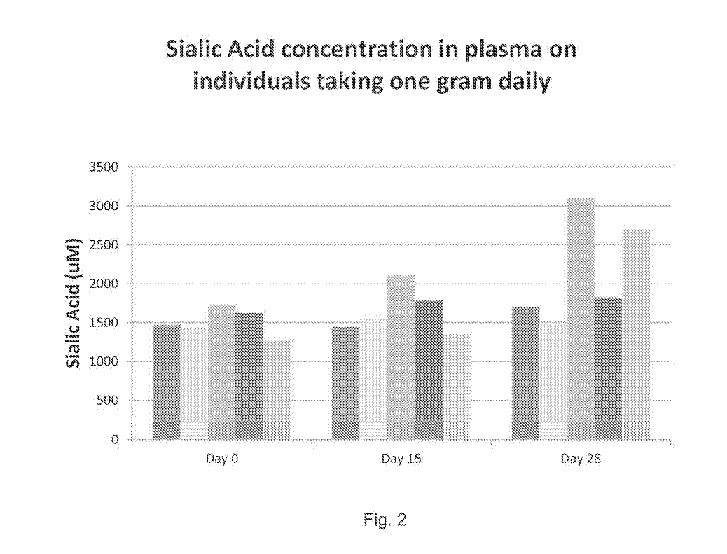

### DETAILED DESCRIPTION

The following definitions are provided to allow for a better comprehension of the invention:

The use of the term “approximately” or “about” provides an additional determined range. The term is defined in the following way. The additional range provided by the term is approximately ±10%. By way of example, but not limitative, if it reads “approximately 40 g”, the exact range is between 36 to 44 grams.

Term “treat” or “treatment” according to this invention refers to a prophylactic treatment, including those subjects at risk of contracting a disease or suspected to have contracted a disease, as well as patients having a disease or disorder related to a deficiency of NeuAc and/or Magnesium.

It is worth mentioning that compositions or nutritional supplements of the invention are not intended to cure a patient suffering from a disease or disorder related to a NeuAc deficiency, but to increase dietary intake of NeuAc which, as should be evident for a skilled artisan, could provide a health benefit to such patients.

Term “effective amount” refers to an amount that provides a nutritional benefit to a subject or patient.

Term “derivative” refers to at least one compound selected from N-acetyl mannosamine (ManNac), at least one or more compounds of the NeuAc biosynthetic pathway, substrates, products, or derivative thereof, or a mixture thereof.

Term “subject” or “patient” refers to a healthy mammal or a mammal at risk of having or suffering a disease or disorder related to a NeuAc and/or Magnesium deficiency.

Surprisingly, inventors from this invention have found that NeuAC in combination with magnesium improve bio-availability and cellular usability of NeuAc. Inventors speculate this beneficial effect is because Magnesium is an essential co-factor for the enzyme responsible for cellular production of NeuAc, as well as numerous other enzymes, thus enhancing the effect of NeuAc. The same benefit is expected for N-acetyl mannosamine (ManNac), a compound of the NeuAc biosynthetic pathway such as sialyllactose, substrates, products, or derivative thereof, or a mixture thereof, when combining with magnesium.

While not wishing to be bound by any theory, inventors believe that benefits of the composition and nutritional supplement of the invention over complications due to neuropathies, hypertension or hypotension or kidney disorders is due to an improvement in the erythrocyte capacity in such cases wherein the erythrocyte charge prevents the correct erythrocyte functionality in capillaries.

As indicated above, the invention relates to compositions comprising NeuAc and a divalent metal ion useful as a nutritional supplement.

Further, the compositions of the invention may also comprise at least one compound selected from the group of ManAc, a compound in the NeuAc biosynthetic pathway or derivative thereof, or a mixture thereof.

Alternatively, the compositions of the invention comprise siallylactose and a divalent metal ion.

The compositions of the invention may further comprise one or more of the following components: a flavoring agent, a sweeter agent, a diluent, a binding agent, a pH adjuster, a stabilizer, and at least one controlled release polymer to permit an extended, controlled or sustained delivery of active ingredients. Said components may be present in the composition from about 0.01 mg to about 35 grams.

Flavoring agents could be selected from one or more of the following: pineapple flavor, parsley flavor, strawberry flavor, orange flavor, lemon flavor, gooseberry flavor, and conventional flavors already know in the art. Flavoring agents could be natural flavoring or synthetic flavorings. In some preferred embodiments of the invention, fruit pulp, such as pineapple pulp or parsley pulp, could be used as flavoring agents.

Sweeteners could be selected from one or more of the following: natural sweeteners obtained from sugarcane, sugar beet or fruits; or artificial sweeteners such as sucralose, acesulfame, Aspartame, Cyclamate, Saccharin, Stevioside, Neohesperidine, Aspartame-Acesulfame Salt.

Diluents could be selected from water, preferably purified water.

The binding agent could be selected from one or more of the following: ethyl cellulose, saccharides, gelatins, pregelatinized starches, microcrystalline cellulose, hydroxypropylcellulose and cellulose ethers, as well as polyvinylpyrrolidone (PVP).

The composition of the invention may comprise from about 0.001 mg to about 15 g, preferably from about 200 mg to about 12 g, more preferably from about 500 mg to about 6 g of one or more of the following: NeuAc, N-acetyl mannosamine (ManNac), a compound of the NeuAc biosynthetic pathway, substrates, products, or derivative thereof, or a mixture thereof. In a preferred embodiment, the composition of the invention comprises about 1 g of one or more of these compounds.

In a preferred embodiment, the ratio of NeuAc or a derivative thereof to Magnesium is 1:0.0276 such that, for instance, when 1 g of NeuAc is present in the composition, magnesium will amount 0.0276 g.

The invention further provides methods of treating and preventing NeuAc deficiencies utilizing these compositions, permitting stable and steady day and nighttime NeuAc concentrations without high concentration spikes across a broad in multiple tissues.

To overcome the common problems due to poor absorption of of NeuAc, the compositions of the invention are formulated as sustained/controlled/extended release formulations to keep blood levels of NeuAc high throughout the 24 hours to sustain chronic benefit and incorporation of oral NeuAc in glycans.

The compositions can be made to advantageously using coating systems to protect against both bacterial and stomach acid breakdown. The compositions can be made to improve absorption by using hydrophobic, liposomal, and/or microsomal, nanoparticles to benefit of other absorption mechanisms (e.g. lymphatic system).

Examples of controlled release polymers that may be used according to the invention are the following: ethylcellulose such as that sold under the trademark Aquarius® SRX, a natural polymer (e.g., polysaccharide or protein), a modified natural polymer, and/or synthetic polymer. The controlled release polymer may be further selected from, for example, a hydrophobic polymer, hydrophilic polymer, hydrogel, soluble polymer, biodegradable polymer, nonbiodegradable polymer, and/or mucoadhesive polymer, preferably the controlled release polymer is ethylcellulose.

In some embodiments, the polymer is a hydrophobic polymer, which could be selected from polyethylene, polyvinyl chloride, ethyl cellulose or acrylate polymers and their copolymers.

In certain embodiments, the polymer is a biodegradable polymer, which could be selected from the group comprising polylactic acid (PLA), polyglycolic acid (PGA), poly(lactic/glycolic acid) (PLGA), polycaprolactone (PCL), polyanhydrides, or polyorthoesters.

In some embodiments, the extended release composition comprises ProCR hypromellose, which is a water-swellable, pH independent polymer. In some embodiments, the extended release composition further comprises an anionic, pH-dependent, gel-forming copolymer (e.g., an alginate salt). In some embodiments, the extended release composition further comprises a hydrocolloid polymer (e.g., carrageenan). In some embodiments, the extended release composition comprises a water-swellable, pH independent polymer (e.g. hypromellose), an anionic, pH-dependent, gel-forming copolymer (e.g., an alginate salt) and a hydrocolloid polymer (e.g., a carrageenan).

The nutritional supplement for humans or animals of the invention also permits to increase glycosilation.

The nutritional Supplement is provided in all cases where it is desired to have a diet focused on nutritional treatment or prevention or nutritional support of healthy individuals and subjects with various ailments including but not limited to neurological disorders, neurodegenerative disorders, cognitive impairment or behavior, e.g. elderly, young children and nutritional deterioration, and others at risk of sub-optimal NeuAc cellular production or dietary bio-availability. Further, magnesium will likely provide additional biological benefits for a subject such as those listed above.

The nutritional supplement of the invention can be used by humans and animals to improve nutritional state, and biological functions. When animals intake the nutritional supplement of the invention, an improvement in the nutritional value of milk/meat and other food products is obtained.

As mentioned previously, the compositions of the invention may provide to a subject between about 0.001 mg to 15 g/day of NeuAc.

The extended release compositions of the invention may further comprise an excipient, an antioxidant, a lubricant, a colorant, a disintegrant, and the like.

The excipient may be selected from the group consisting of lactose, microcrystalline cellulose, corn starch, potato starch, wheat starch, sucrose, D-mannitol, precipitated calcium carbonate, dextrin, pre-gelatinized starch, and combinations thereof.

The nutritional supplement may comprise any of the compositions above described.

The nutritional supplement is in the form of powder or granules. Even in this form, the nutritional supplement will provide a controlled delivery of NeuAc or derivative due to the presence of the controlled release polymer.

The nutritional supplement is in the form of a tablet designed for controlled release.

In some embodiments, the controlled release polymer may be present in the composition of the invention from about 0.01 mg to about 30 grams.

In some embodiments, the daily doses vary depending on the age.

In some embodiments, the nutritional supplement is administered in doses ranging for instance from about 0.1 mg-10 g/day for children between 1-16 years old and in doses ranging for instance from about 0.1 mg-12 g/day for adults.

The nutritional supplement as described herein may be formulated into preparations in solid, semi-solid, liquid or gaseous forms, such as tablets, capsules, powders, granules, ointments, solutions, suppositories, injections, inhalants, gels, microspheres, and aerosols. In some embodiments, the nutritional supplements of the invention are formulated for administration by a variety of routes including oral, parenteral (including subcutaneous, intravenous, intramuscular and intraperitoneal), rectal, dermal, transdermal, intrathoracic, intrapulmonary and intranasal (respiratory) routes.

For oral administration, the nutritional supplement of the invention herein can be administered in solid dosage forms, such as capsules, tablets, granules and powders, or in liquid dosage forms, such as elixirs, syrups, and suspensions.

In some embodiments, the compositions of the nutritional supplement comprise an enteric-coating. Numerous types of acid resistant enteric coatings are available. Examples of the acid resistant coatings include cellulose acetate phthalate, polyvinyl acetate phthalate, shellac, an acrylic acid homopolymer or copolymer, a methacrylic acid homopolymer or copolymer, cellulose acetate trimellitate, hydroxyl propyl methylcellulose phthalate or a combination of thereof. A number of copolymers of methacrylic acid are known in the art and are comercially available. Examples of such polymers are copolymers of methylmethacrylate and methacrylic acid and copolymers of ethylacrylate and methacrylic acid, and sold under the tradename Eudragit (Rohm GmbH & Co. KG): examples include Eudragit® L 100-55, Eudragit® L 30D-55, Eudragit® L 100, Eudragit® S 100-55 and Eudragit® PS 30D. In some embodiments, the enteric coating comprises one or more of titanium dioxide, polydextrose, hypromellose, triacetin and macrogol/PEG.

An enteric coating can also be a time-release coating. The time-release coatings are degraded away at a relatively constant rate until the coatings dissolve sufficiently for the time-release coatings to rupture. Thus, the time required for the rupture of the enteric coatings is largely time-dependent (i.e., thickness), and largely pH independent. Examples of time-release coating materials include cellulose acetate, cellulose acetate butyrate, cellulose acetate propionate, EC, and copolymers of acrylate and methacrylates with quaternary ammonium groups such as Eudragit® RL and Eudragit® RS and Eudragit® NE30-D.

The extended release compositions can be further subjected to a process of film coating. For the film coating agent, an enteric or non-enteric film coating agent may be used, and the enteric film coating agent can be cellulose acetate phthalate (CAP), polyvinyl acetate phthalate (PVAP), a methacrylate polymer (Eudragit L, S), or the like, while the non-enteric film coating agent can be hydroxypropylcellulose (HPC), MC, EC, HPMC, povidone, PVA, CA, shellac, or the like. The process of coating can be performed by, for example, a pan coating method, a fluidized bed coating method, a compression coating method, or the like.

Coated tablets of the extended release composition may be prepared in various sizes. For example, the coated tablets may have a length of about 16-20 mm, a width of about 7-12 mm and a thickness of about 5-8 mm. In some embodiments, the coated tablets have a length of about 17.7 mm, a width of about 9.1 mm and a thickness of about 6.7 mm. In some embodiments, the coated tablets have a length of about 19.3 mm, a width of about 9.7 mm and thickness of about 8.0 mm.

In preferred embodiments, the nutritional supplement of the invention is capable of delivering to an individual in need thereof NeuAc or a derivative for a period from about 1 hour to about 24 hours. The compositions of the invention and/or nutritional supplement may be formulated for parenteral administration (e.g., by injection, for example, bolus injection or continuous infusion) and may be presented in unit dose form in ampoules, pre-filled syringes, small volume infusion containers or in multi-dose containers. The nutritional supplement may be in the form of suspensions, solutions, or emulsions in oily or aqueous vehicles.

For topical administration, the nutritional supplement may be formulated as is known in the art for direct application to a target area. For example, as creams, milks, gels, dispersion or microemulsions, lotions thickened to a greater or lesser extent, impregnated pads, ointments or sticks, aerosol compositions (e.g., sprays or foams), soaps, detergents, lotions or cakes of soap. Other conventional forms for this purpose include wound dressings, coated bandages or other polymer coverings, ointments, creams, lotions, pastes, jellies, sprays, and aerosols. The compositions of the invention and/or nutritional supplement may be formulated as patches or bandages for dermal administration. Ointments and creams may, for example, be formulated with an aqueous or oily base with the addition of suitable thickening and/or gelling agents. Drops, such as eye drops or nose drops, may be formulated in an aqueous or non-aqueous base. Liquid sprays are conveniently delivered from pressurized packs. Drops can be delivered via a simple eye dropper-capped bottle, or via a plastic bottle adapted to deliver liquid contents dropwise, via a specially shaped closure.

In preferred embodiments, the compositions of the invention and/or nutritional supplement may optionally be used in combination with therapeutic agents.

Articles of manufacture comprising the compositions of the invention and/or the nutritional supplement are also within the scope of the invention.

The articles of manufacture or kits comprising: (a) a container comprising the nutritional supplement or composition of the invention showing an extended release; and (b) a package insert with instructions to correct or avoid deficiencies related to NeuAc, or for dietary support of a subject to advance or accelerate the rate of recovery from a disorder related to NeuAc deficiency. Articles of manufacture according to this invention also comprise polyethylene bags comprising the nutritional supplement. Unit dosages comprising the nutritional supplement as described herein are also provided.

The unit dosage forms can be stored in a suitable packaging in single or multiple unit dosages and may also be further sterilized and sealed. For convenience and ease of compliance, the extended release supplement may be delivered in the form of unit dosage forms, which may be intake by a subject. In one variation, the extended release supplement is a solid substance and unit dosage forms thereof may be prepared in the form of tablets, capsules, granules or powders such as powders to be reconstituted, sachets and chewable tablets.

The methods for preparing the compositions of the invention and nutritional supplements are within the scope of the invention.

### EXAMPLES

The following examples are provided by way of illustration only and not by way of limitation.

### Example 1

### Preparation of a Composition According to the Invention Comprising NeuAc, Magnesium and an Extended Release Polymer:

NeuAc (1.0 g), ethylcellulose (0.15 g, Aquarius Control Srx) and magnesium (0.0276 g) are sieved with a mesh No. 18. The resulting sieved mixture is added into a ribbon mixer, wherein is mixed for about 10 minutes. Next, water (0.2 mL) is added and the mixture is granulated. For this step, the solution is divided in equal parts with the object of distribute it in 4 minute-intervals until reaching 20 minutes. Granulation step is continued for about 5 minutes.

The granules are sieved with a mesh No. 14 and, afterwards, granules are dried until a HR from 2.5 to 4.5% is obtained.

Next, the following ingredients are added to the ribbon mixer: pineapple pulp (0.0423 g), parsley pulp (0.0099 g), sucralose (0.0101 g) and pineapple flavoring (0.0101 g) and the mix is continued for about 10 minutes.

The resulting granules are packed in polyethylene bags.

### Example 2

### Administration of the Nutritional Supplement to a Group of Subjects:

The nutritional supplement of the invention prepared as per example 1 was administered to a first group conformed by five subjects. A second group of five subjects was the control group. The first group was administered the nutritional supplement of the invention on a daily basis. The control group was administered glucosamine (1 g) via oral on a daily basis.

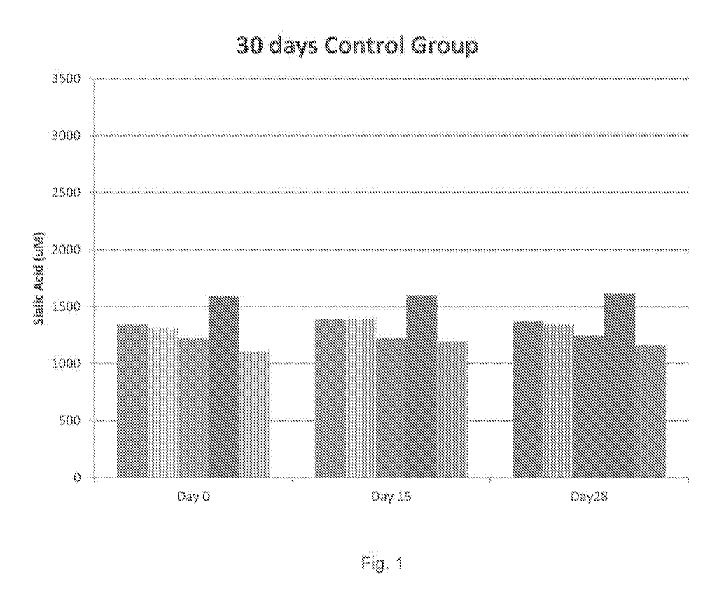

The results obtained are illustrated in Tables I and II and in FIGS. 1 (control group) and 2 (nutritional supplement of the invention). It results evident that subjects of the first group had an increment in the NeuAc levels in comparison with the control group, when taking samples at day 15 and day 28.

TABLE 1
Serum concentrations of NeuAc in control group:

| | | | Sialic acid |
|---|---|---|---|
| | Sample ID | Ave OD | (uM) |
| Control | Plasma Day 0 | 306598 | 1341 |
| Group | Plasma Day 0 | 296510 | 1300 |
| | Plasma Day 0 | 277254 | 1220 |
| | Plasma Day 0 | 367328 | 1594 |
| | Plasma Day 0 | 248999 | 1102 |
| | Plasma Day 15 | 317895 | 1388 |
| | Plasma Day 15 | 361653 | 1394 |
| | Plasma Day 15 | 317546 | 1223 |
| | Plasma Day 15 | 369201 | 1601 |
| | Plasma Day 15 | 309730 | 1193 |
| | Serum Day 28 | 354644 | 1368 |
| | Serum Day 28 | 346649 | 1337 |
| | Serum Day 28 | 321625 | 1243 |
| | Serum Day 28 | 371681 | 1612 |
| | Serum Day 28 | 300625 | 1163 |

TABLE II
Serum concentrations of NeuAc in first group:

| | | | Sialic acid |
|---|---|---|---|
| | Sample ID | Ave OD | (uM) |
| First Group | 1-4 plasma Day 0 | 381573 | 1470 |
| (Nutritional | 2-4 plasma Day 0 | 370685 | 1428 |
| Supplement) | 3-4 plasma Day 0 | 451593 | 1735 |
| | 4-4 plasma Day 0 | 422075 | 1623 |
| | 5-4 plasma Day 0 | 333836 | 1289 |
| | 1-5 plasma Day 15 | 375091 | 1445 |
| | 2-5 plasma Day 15 | 403974 | 1554 |
| | 3-5 plasma Day 15 | 549492 | 2105 |
| | 4-5 plasma Day 15 | 464596 | 1784 |
| | 5-5 plasma Day 15 | 348640 | 1345 |
| | 1-6 plasma Day 28 | 442561 | 1700 |
| | 2-6 plasma Day 28 | 385083 | 1483 |
| | 3-6 plasma Day 28 | 813373 | 3104 |
| | 4-6 plasma Day 28 | 476221 | 1828 |
| | 5-6 plasma Day 28 | 704203 | 2691 |

It should be noted that any of the subjects suffered toxical effects.

Although illustrative embodiments of the present invention have been described herein, it should be understood that that the invention is not limited to those disclosed, and that various other changes or modifications can be made by one skilled in the art without departing from the scope or spirit of the invention.

1. A composition useful as nutritional supplement permitting to increase serum and cellular NeuAc content in a subject comprising: acetylated neuraminate (NeuAc) and/or a compound selected from N-acetyl mannosamine (ManNac), a compound of the NeuAc biosynthetic pathway, substrates, products, or derivative thereof, or a mixture thereof; and a divalent metal ion.

2. The composition of claim 1, wherein divalent metal ion is magnesium.

3. The composition of claim 1, wherein the compound of the NeuAc biosynthetic pathway is siallylactose.

4. The composition of claim 1, wherein the composition comprises acetylated neuraminate (NeuAc), siallylactose and a divalent metal ion.

5. The composition of claim 1, wherein the composition comprises siallylactose and a divalent metal ion.

6. The composition of claim 1, further comprising a flavoring agent, a sweeter agent, a diluent.

7. The composition of claim 1, further comprising at least one controlled release polymer.

8. The composition of claim 7, wherein the controlled release polymer may be selected from: ethylcellulose, a natural polymer, a modified natural polymer, a synthetic polymer, a hydrophobic polymer, a hydrophilic polymer, a hydrogel, a soluble polymer, a biodegradable polymer, a nonbiodegradable polymer, and a mucoadhesive polymer.

9. The composition of claim 6, wherein flavoring may be selected from natural flavoring or synthetic flavorings.

10. The composition of claim 6, wherein natural sweeteners may be selected from natural sweeteners or artificial sweeteners.

11. The composition of claim 6, wherein the diluent is water.

12. The composition of claim 6, wherein the binding agent could be selected from one or more of the following: ethyl cellulose, saccharides, gelatins, pregelatinized starches, microcrystalline cellulose, hydroxypropylcellulose and cellulose ethers, as well as polyvinylpyrrolidone (PVP).

13. The composition of claim 1, wherein the NeuAc is present in the composition from about 200 mg to about 12 g.

14. A nutritional supplement to increase serum and or cellular NeuAc content in a subject, comprising the composition of claim 1.

15. A method for treating and preventing NeuAc deficiencies comprising administering an effective amount of a composition comprising acetylated neuraminate (NeuAc), and/or siallylactose and a divalent metal ion to a subject in need thereof.

16. The method of claim 15, wherein the divalent metal ion is magnesium.

17. An article of manufacture comprising a composition as claimed in claim 1.
